(19) United States
(12) Patent Application Publication
(10) Pub. No.: US 2008/0234666 A1
(43) Pub. Date: Sep. 25, 2008

(54) SURGICAL LASER SYSTEM CONTROL ARCHITECTURE

(76) Inventors: Michael J. Yadlowsky, Irvine, CA (US); Thomas James Matheson, Mission Viegjo, CA (US); Xiang Simon Han, Laguna Nigel, CA (US)

Correspondence Address: ALCON, IP LEGAL, TB4-8, 6201 SOUTH FREEWAY, FORT WORTH, TX, 76134, US

(21) Appl. No.: 12/052,443

(22) Filed: Mar. 20, 2008

Related U.S. Application Data
(60) Provisional application No. 60/895,900, filed on Mar. 20, 2007.

(51) Int. Cl. A61B 18/20

(52) U.S. Cl. 606/4

(57) ABSTRACT

Embodiments of the present invention provide a surgical laser system having a control architecture operable to enable the surgical laser to be controlled from a user interface local to the laser system (e.g., the laser system front panel) and from a remote device. According to one embodiment of the present invention, a surgical laser unit operable to implement one set of functionality may be coupled to an advanced control unit such that the surgical laser unit may be controllable by the advanced control unit. The advance control unit may be a surgical system such as a vitreoretinal surgical system or other such surgical system.

### CROSS-REFERENCE TO RELATED APPLICATIONS

This application claims priority under 35 U.S.C. §119 to U.S. Provisional Patent Application No. 60/895,900 filed Mar. 20, 2007, the entire contents of which are incorporated herein by reference.

### TECHNICAL FIELD OF THE INVENTION

The present invention relates to surgical devices. More particularly, the present invention relates to surgical laser systems. Even more particularly, the present invention relates to surgical laser system software control architecture operable to provide for consistent control of a surgical laser from various user interfaces.

### BACKGROUND

The human eye can suffer a number of maladies causing mild deterioration to complete loss of vision. While contact lenses and eyeglasses can compensate for some ailments, ophthalmic surgery is required for others. Generally, ophthalmic surgery is classified into posterior segment procedures, such as vitreoretinal surgery, and anterior segment procedures, such as cataract surgery. More recently, combined anterior and posterior segment procedures have been developed.

The surgical instrumentation used for ophthalmic surgery can be specialized for anterior segment procedures or posterior segment procedures or can support both. In any case, the surgical instrumentation often implements a host of functionality which may be used in the implementation of a wide variety of surgical procedures.

Laser surgery to the retina is the standard of care in the treatment of numerous ophthalmic diseases. Diseases treated by laser photocoagulation include proliferative diabetic retinopathy, diabetic macular edema, cystoid macular edema, retinal vein occlusion, choroidal neovascularization, central serous chorioretinopathy, retinal tears, and other lesions. Depending on the complexity of the case, these procedures may be performed as part of a complex procedure in a hospital operating room (“OR”) with a large amount of infrastructure, or as a single procedure in a less costly treatment area within a surgeon's clinical office. The equipment needs of these two environments can differ substantially. In an office-based treatment facility, surgeons typically prefer a compact, portable stand-alone laser unit. In an OR when vitreous surgery needs to be performed, it can save space and cost to integrate a surgical laser with a vitreoretinal surgical system. In such combined systems it may be preferable for a surgeon to be able to control the laser using the same control interface used to control the vitreoretinal surgical system.

Prior art ophthalmic surgical systems have not been able to successfully integrate the software control system architecture of a surgical laser across various platforms. Even when a surgical laser is integrally combined as part of a more complex surgical system, such as a vitreoretinal surgical system, the user interface for the laser is typically separate from that of the rest of the surgical system with which it is combined. However, whether used as a standalone device or integrated with a surgical system, a laser is more easily and more safely operated if control of the surgical laser is consistent across various configurations to accommodate use of the laser in different modes. Typically, switching back and forth in this manner is complicated by the fact that the user interface design and the type of controls used are different between the stand alone laser unit and the host surgical system. Furthermore, maintaining coordinated behavior can be difficult as the firmware of either system is upgraded.

Therefore, a need exists for a surgical laser system control architecture that will enable a surgical laser to be controlled either from a user interface local to the laser system (e.g., the laser system front panel) or from a remote device, such as a host system (e.g., a vitreoretinal surgical system) user interface.

### SUMMARY OF THE INVENTION

Embodiments of the present invention meet this need and others. Embodiments of this invention provide a surgical laser system having a control architecture that will enable control of the surgical laser from either a user interface local to the laser system (e.g., the laser system front panel) or from a remote device, such as a host vitreoretinal system user interface.

According to one embodiment of the present invention, a surgical laser unit operable to implement one set of functionality may be coupled to an advanced control unit such that the surgical laser unit may be controllable by the advanced control unit. The advance control unit may be a surgical system, such as a vitreoretinal surgical system, or other ophthalmic surgical system.

Thus by integrating functionality of the surgical laser system, as regards the software control architecture and perhaps mechanical components, embodiments of the present invention provide the advantage that a basic laser unit may be easily interfaced to an external control device. The learning curve required to utilize the basic unit may thus be carried over to a more complicated external unit, allowing for a common user interface whether a surgical laser unit is used as a standalone device, or controlled via an external device such as a vitreoretinal surgical system.

Similarly, embodiments of the present invention can provide the advantage of a basic laser unit that can utilized in the implementation of more complex functionality, thus eliminating the need to duplicate the functionality or capabilities of the basic unit when implementing this advanced functionality. This may be advantageous to users of surgical laser systems as they may able to purchase a basic unit at a lower initial price and have a cost effective upgrade path to advanced functionality that does not render the basic unit redundant.

These, and other, aspects of the invention will be better appreciated and understood when considered in conjunction with the following description and the accompanying drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE FIGURES

A more complete understanding of the present invention and the advantages thereof may be acquired by referring to the following description, taken in conjunction with the accompanying drawings in which like reference numbers indicate like features and wherein:

FIG. 1 is a diagrammatic representation of one embodiment of a surgical laser system in accordance with the present invention;

FIG. 2 is a diagrammatic representation of one embodiment of a surgical laser system coupled to a control unit in accordance with the present invention;

FIG. 3 is a diagrammatic representation of one embodiment of a surgical laser system integrated within a vitreoretinal surgical system in accordance with the teachings of this invention;

FIG. 4 is a diagrammatic representation of one embodiment of a graphical user interface of an embodiment of the present invention;

FIG. 5 is a diagrammatic representation of one embodiment of control architecture 400 for a stand alone surgical laser unit 100 of the present invention;

FIG. 6 is a diagrammatic representation of an embodiment of the control architecture 400 for a surgical laser unit 100 of the present invention integrated with an advanced control unit 200; and

FIG. 7 is a diagrammatic representation of an embodiment of the control architecture 400 for a surgical laser unit 100 tethered (coupled) to an advanced control unit 200 in accordance with the present invention.

### DETAILED DESCRIPTION

Preferred embodiments of the invention are illustrated in the FIGURES, like numerals being used to refer to like and corresponding parts of the various drawings.

Embodiments of this invention provide a surgical laser system and a control architecture that will enable control of the surgical laser from a user interface local to the laser system (e.g., the laser system front panel) and from a remote or host device, such as a vitreoretinal system user interface. According to one embodiment of the present invention, a surgical laser system (e.g. a basic unit) operable to implement a set of functionality, such as those functions associated with a set of main laser parameter settings, and having some customizable features, system statistics and diagnostics, etc., may be coupled to another unit (e.g. an advanced control unit) such that the basic unit may be controllable by the advanced control unit to implement, or allow to be implemented, the basic unit set of functionality or a more complex functionality. The implemented functionality can include pre-operation picture viewing, creating custom marked treatment pictures, creating and printing patient record, advanced customizations, doctor login to activate custom settings, creating and firing custom laser pulse sequences, E-connectivity through an Ethernet port or wireless communication of diagnostics, statistics, service needs or to upload software upgrades, wireless RFID check in for the doctor and customer, etc. The functionality of the surgical laser unit, whether in standalone or coupled mode, can be controlled through a common laser control system that can be accessed via either the basic unit or the advanced control unit user interfaces. In this way, commands from either interface can be translated to achieve a desired task regardless of the specific user interface implementation.

The embodiments of this invention can thus provide for the control of a base surgical laser system by another unit, such as vitreoretinal surgical system or other ophthalmic surgical system (i.e. remote controlled) to implement the same and potentially more functionality than the base surgical laser system is capable of implementing as a standalone device. By allowing the basic unit's functionality to be controlled locally or via an external unit, the basic unit may be streamlined with regards to both cost and size, and the learning curve required to utilize the basic unit may be reduced relative to an “all-in-one” unit, allowing the basic unit to be simpler and more cost-effective than an “all-in-one” unit while allowing for future expansion. Because a basic unit may be utilized in the implementation of more complex functionality (via a coupled advanced control unit), there is no need to duplicate the functionality or capabilities of the basic unit when implementing other functionality via an external device. This may be advantageous to users of such surgical laser systems as they may then purchase a basic unit at a lower initial price and have a cost effective upgrade path to advanced functionality that does not render the basic unit redundant.

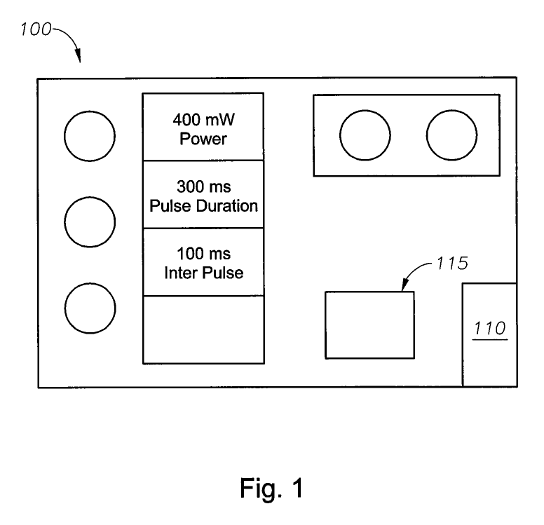

FIG. 1 is a diagrammatic representation of one embodiment of a surgical laser unit with a basic functionality. Basic surgical laser unit 100 can comprise a laser source and associated control software such that basic surgical laser unit 100 is operable to implement a set of functionality such as that discussed above.

In one embodiment, basic surgical laser unit 100 can comprise a laser source similar to that of the Alcon EyeLite Photocoagulator manufactured by Alcon Laboratories, Inc., of Fort Worth, Tex., and associated software operable to provide a set of functionality using basic surgical laser unit 100. Basic surgical laser unit 100 may also comprise a communications port 110, allowing basic surgical laser unit 100 to be communicatively coupled to an external advanced control unit such that basic surgical laser unit 100 may be controlled by the advanced control unit (i.e. remotely controlled) to implement the same and/or additional functionality (i.e. a more advanced or different set of functionality than may be implemented with basic surgical laser unit 100 alone). An advanced control unit can be, for example, a vitreoretinal or other surgical system that may be used in conjunction with a surgical laser. Basic surgical laser unit 100 can also comprise a user interface 115, which can comprise a touch screen, mouse, keyboard or other user input device. User interface 115 is used by an operator to select or control the functionality of basic surgical laser unit 100.

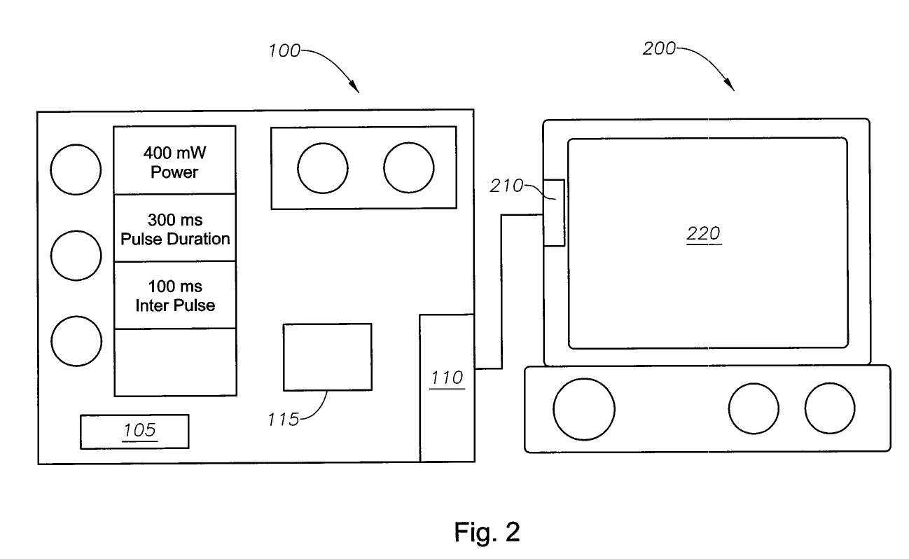

FIG. 2 is a diagrammatic representation of an embodiment of a basic surgical laser unit 100 coupled to an advanced control unit 200. Basic surgical laser unit 100 and advanced control unit 200 can be coupled to one another through communications ports 110 and 210 on basic surgical laser unit 100 and advanced control unit 200, respectively. Advanced control unit 200 can include software (e.g. computer-executable instructions on a computer readable medium) and a microprocessor operable to allow advanced control unit 200 to control basic surgical laser unit 100 or components thereof (e.g. the laser source 105 of basic surgical laser unit 100) to implement the same and/or a different set of functions (e.g. more or advanced features) than basic surgical laser unit 100 is operable to implement in a standalone configuration.

Thus, in some embodiments, the software and/or microprocessor of advanced control unit 200 can also implement (e.g. duplicate) the functionality of basic surgical laser unit 100, such that advanced control unit 200 can control basic unit 100 to implement both the basic set of functionality and an additional set of functionality (e.g. the set of functionality which can be implemented utilizing advanced control unit 200 and basic surgical unit 100 is a superset of the functionality which can be implemented using basic surgical unit 100 in a standalone configuration). Advanced control unit 200 can also comprise user interface 220, which can also comprise a touch screen, keyboard, mouse or other user input device as know to those having skill in the art. User interface 220 can be used to select or control the functionality of either, or both, of advanced control unit 200 and basic surgical laser unit 100.

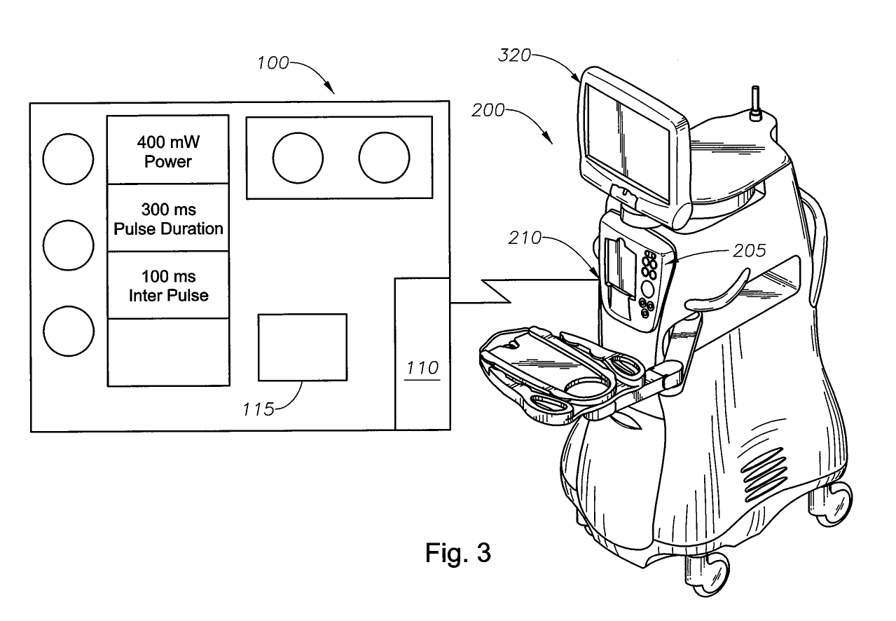

Moving to FIG. 3, another arrangement by which the functionality of a basic surgical laser unit 100 can be increased by coupling it to an advanced control unit 200 is depicted. In this embodiment, advanced control unit 200 comprises a surgical console similar to the Series 2000® Legacy® cataract surgical system, the Accurust 400VS surgical system, and/or the Infiniti™ Vision System surgical system, all available from Alcon Laboratories Inc. of Fort Worth, Tex., and can include a connection panel 205 used to connect various tools and consumables to the surgical console. The connection panel 205 can include, for example, a coagulation connector, balanced salt solution receiver, connectors for various hand pieces and a fluid management system (“FMS”) or cassette receiver. Surgical console 200 can also include a variety of user friendly features, such as a foot pedal control (not shown) and other such features. Advanced control unit 200 may also include swivel monitor 320 which can comprise a touchscreen user interface and can be positioned in a variety of orientations for whoever needs to see the touch screen of the swivel monitor 320. Swivel monitor 320 can swing from side to side, as well as rotate and tilt. A graphical user interface (“GUI”) that allows a user to interact with surgical console 200 can be provided or presented on the touch screen of swivel monitor 320.

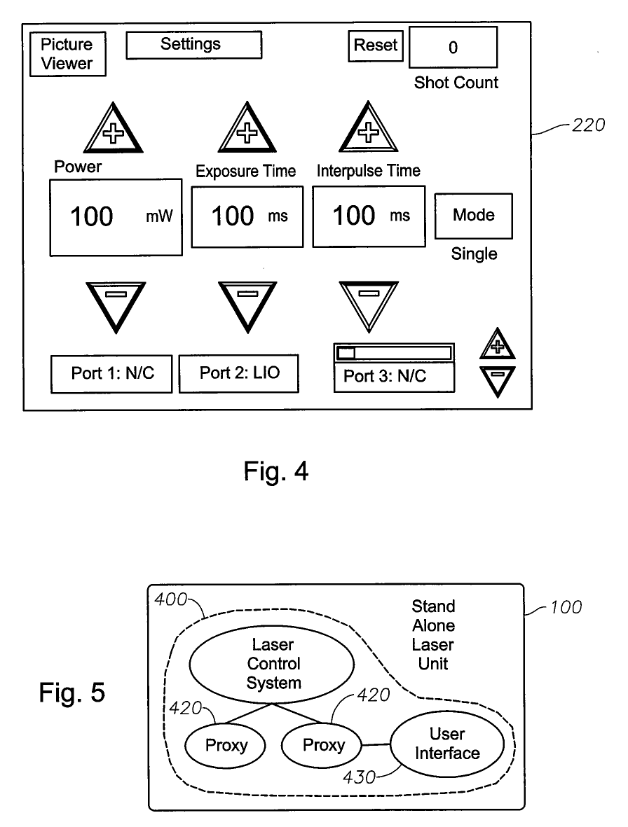

As discussed above, advanced control unit 200 may comprise communications port 210, through which advanced control unit 200 may be coupled to basic surgical laser unit 100 (e.g. advanced control unit 200 and basic surgical laser unit 100 may communicate through communication ports 110 and 210) and advanced control unit 200 can include software and/or a microprocessor such that advanced control unit 200 is operable to control basic surgical laser unit 100 to implement the same and/or a different set of features than basic surgical laser unit 100 is operable to implement in a standalone configuration. Thus, in one embodiment, utilizing a GUI provided on the touch screen of swivel monitor 320, an operator may control the combination of advanced control unit 200 and basic surgical laser unit 100 to implement additional functionality that basic surgical laser unit 100 may be incapable of implementing in a standalone configuration. One example of such a graphical user interface is depicted in FIG. 4. Alternatively, advanced control unit 200 may comprise, as an integral part, a basic surgical laser unit 100 that is built-in as part of advanced control unit 200. These embodiments are functionally the same and differences exist mainly as to how the two units are physically connected.

It will be apparent to those skilled in the art that the coupling between basic surgical laser unit 100 and advanced control unit 200 may be accomplished via any suitable coupling mechanism and/or protocol. More particularly, communication between the basic surgical laser unit 100 and advanced control unit 200 may occur via a wired or wireless interface. Advanced control unit 200 can, for example, have a set of slots such that the basic surgical laser unit 100 may “plug-in” to a spot in the chassis of an advanced control unit 200 (for example, through a backplane interface present in advanced control unit 200). In one particular embodiment, communication ports 110 and 210 may be Ethernet ports, as will be known to those having ordinary skill in the art.

It may be imagined, however, that in many cases basic surgical laser unit 100 and advanced control unit 200 may be sensitive devices, and may comprise components (e.g. laser sources) that could pose a danger if they are improperly used. Consequently, it may not be desirable to utilize a standard protocol which can be easily learned and taken advantage of to manipulate basic surgical laser unit 100 or advanced control unit 200 without proper training and/or authorization. Therefore, in some embodiments a standard connector may be used (e.g. an Ethernet connector) for communications ports 110 and 210; however, a variation may be implemented on this standard connector to implement proprietary communication between basic surgical laser unit 100 and advanced control unit 200. For example, one or more pins of the Ethernet connectors comprising communications port 110 and 210 may be scrambled (e.g. lines between the two communication ports 110 and 210 may connect to pins in locations other than those specified according to the standard Ethernet protocol, or pins of communication ports 110 and 210 can be used for non standard purposes). In addition to preventing unauthorized control of basic surgical laser unit 100 and/or advanced control unit 200, these types of scrambling arrangements may allow basic surgical laser unit 100 or advanced control unit 200 to detect the coupling of unauthorized or incompatible devices, or attempts at control or communication, and take appropriate remedial action, such as logging the improper access, shutting down, sounding an alarm, etc.

FIG. 5 is a diagrammatic representation of one embodiment of the control architecture 400 of a stand alone basic surgical laser unit 100. Control architecture 400 is designed in accordance with the present invention for interfacing to external control devices, such as an advanced control unit 200 discussed above. Control architecture 400 comprises laser control system 410, proxies 420 and user interface 430 (e.g., the software component of user interface 115). Control architecture 400 comprises computer executable software instructions operable to perform at least some of the functions described herein. Control architecture 400 provides for laser control system 410 being operable to be used with multiple devices, including the local user interface 430 of basic surgical laser unit 100, and one or more external devices such as advanced control unit 200, that communicate via the same command set and similar interface.

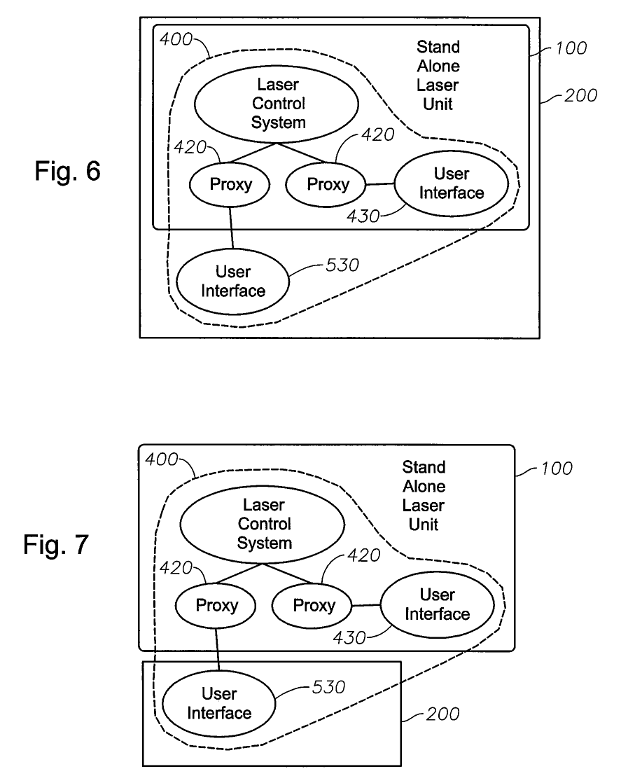

FIG. 6 is a diagrammatic representation of an embodiment of the control architecture 400 of a stand alone basic surgical laser unit 100 integrated with an advanced control unit 200. In this embodiment, user interface 530 (e.g., the software component of use interface 220) of advanced control unit 200 interfaces with laser control system 410 via a proxy 420 to enable control and operation of basic surgical laser unit 100 from advanced control unit 200. In a similar manner, FIG. 7 is a diagrammatic representation of an embodiment of control architecture 400 for a basic surgical laser unit 100 tethered (coupled) to an advanced control unit 200 (as opposed to being integrated within an advanced control unit 200) for unified user interface and control. In this embodiment, user interface 530 of advanced control unit 200 can interface with laser control system 410 of basic surgical laser unit 100 via proxy 420 to enable control and operation of basic surgical laser unit 100 from advanced control unit 200 in a similar manner to the integrated embodiment of FIG. 6.

Multiple physical interfaces, such as Ethernet, RS 232 or an address/data bus (or other interfaces as discussed above and/or known to those having skill in the art) can be supported by the embodiments of the present invention between the user interfaces and the laser control system 410. A software proxy 420 can be used to receive commands from a physical interface driver and deliver the commands to the laser control system 410. Examples of such commands include commands to change laser power, pulse width, inter-pulse time, aiming beam power, etc. Commands specific to a user interface (e.g., user interfaces 430, 530), such as preferences that determine the appearance or behavior of the user interface, do not need to be included since they are local to the user interface itself. Embodiments of the present invention thus can provide a software architecture and command set structure that allow consistent control of a surgical laser system (or any such electronic system) from a local user interface (e.g., user interface 430) or a remote control system (e.g., user interface 530 of advanced control unit 200).

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention as detailed in the following claims.

1. A remotely controllable surgical laser system, comprising:
a surgical laser unit comprising a laser control system operable to implement a first set of functionality; and
a communication port, wherein the surgical laser unit is operable to be controlled through the communication port to implement a second set of functionality, wherein the second set of functionality can be the same as the first set of functionality.

2. The remotely controllable surgical laser system of claim 1, wherein the communications port is one of a wired or a wireless communications port.

3. The remotely controllable surgical laser system of claim 1, wherein the communications port is one of an Ethernet port, an RFID port, and an RS232 port.

4. The remotely controllable surgical laser system of claim 1, wherein the first set of functionality comprises a set of surgical laser unit operating parameter controls.

5. The remotely controllable surgical laser system of claim 1, wherein the second set of functionality comprises one or more functions not included in the first set of functionality.

6. The remotely controllable surgical laser system of claim 1, further comprising a user interface for selecting a function from the first set of functionality.

7. The remotely controllable surgical laser system of claim 1, further comprising a secondary control unit operable to control the surgical laser unit through the communication port, wherein the secondary control unit comprises a second communication port coupled to the surgical laser unit communication port.

8. The remotely controllable surgical laser system of claim 7, wherein the secondary control unit interfaces with the laser control system to implement the second set of functionality.

9. A surgical laser system, comprising:
a surgical laser unit having a first communication port and operable to implement a first set of functionality;
a control unit having a second communication port coupled to the first communication port of the surgical laser system, wherein the control unit is operable to control the surgical laser unit to implement a second set of functionality, wherein the first and second sets of functionality can be the same.

10. A surgical laser control architecture, comprising:
a laser control system for implementing a first set of functionality,
a local user interface for selecting a function from the first set of functionality; and
two or more proxies, for operably coupling the local user interface and a remote user interface to the laser control system to transmit a function selection to the laser control system.

11. The surgical laser control architecture of claim 10, further comprising a remote user interface operably coupled to one of the two or more proxies for selecting a function from a second set of functionality, which can be the same as the first set of functionality, wherein the selected function is transmitted to the laser control system for implementation.

12. The surgical laser control architecture of claim 11, wherein the remote user interface is operably coupled to the respective proxy via a communications port.

13. The surgical laser control architecture of claim 11, wherein the local user interface, laser control system and proxies are included within a local laser system and wherein the remote user interface is included within a remote surgical system.

14. The surgical laser system of claim 13, wherein the remote surgical system is a vitreoretinal surgical system and wherein the local laser system is an ophthalmic surgical laser.
